(19) United States
(12) Patent Application Publication
(10) Pub. No.: US 2021/0086351 A1
(43) Pub. Date: Mar. 25, 2021

(54) SOFT ROBOTIC TOOLS WITH SEQUENTIALLY UNDERACTUATED MAGNETORHEOLOGICAL FLUIDIC JOINTS

(71) Applicant: The Board of Trustees of the University of Alabama, Tuscaloosa, AL (US)

(72) Inventors: Amanda Shannon Koh, Tuscaloosa, AL (US); Xuefeng Wang, Tuscaloosa, AL (US)

(21) Appl. No.: 17/025,749

(22) Filed: Sep. 18, 2020

Related U.S. Application Data
(60) Provisional application No. 62/902,446, filed on Sep. 19, 2019.

(51) Int. Cl. B25J 9/10; B25J 9/16; B25J 19/00; B25J 9/14; B25J 18/06; B25J 9/00

(52) U.S. Cl. CPC B25J 9/1075; B25J 9/1615; B25J 19/0045; H01F 1/447; B25J 18/06; B25J 9/106; B25J 9/0012; B25J 9/14

(57) ABSTRACT

A soft robotic tool may include a plurality of rigid links, a plurality of magnetorheological fluid soft joints, and a plurality of tendons. The rigid links may be disposed in series. Each magnetorheological fluid soft joint may be disposed between a pair of the rigid links. Each magnetorheological fluid soft joint may include a capsule containing a magnetorheological fluid, and an inductive coil disposed around the capsule. The tendons may extend along a length of the soft robotic tool. Each tendon may be attached to each of the rigid links.

### CROSS-REFERENCE TO RELATED APPLICATIONS

This application claims priority to and the benefit of U.S. Provisional Patent Application No. 62/902,446, filed on Sep. 19, 2019, the disclosure of which is expressly incorporated herein by reference in its entirety.

### FIELD OF THE DISCLOSURE

The present disclosure relates generally to robotics and more particularly to soft robotic tools with sequentially underactuated magnetorheological fluidic joints.

### BACKGROUND OF THE DISCLOSURE

Robotic tools may be used in various applications for performing certain tasks or procedures either autonomously or under the guidance of a human operator. For example, in the medical field, a trained clinician may use robotic tools to perform a medical procedure, such as minimally invasive surgery (MIS). In recent years, MIS techniques have become increasingly popular in view of benefits including, for example, smaller incisions, reduced recovery time, lower medical costs, and reduced infection risks. A robotic platform for MIS typically may include one or more surgical tools, navigation systems, and imaging systems configured for performing a desired procedure. An MIS procedure generally may include inserting a flexible tube mounted with one or more tools into the body of a patient and navigating an anatomical pathway to reach buried, diseased, or injured tissue. In some instances, the anatomical pathway may be complex, including non-linear portions, multiple branches, and/or changes in diameter that must be navigated to reach the target tissue. Challenges in navigating such a complex pathway often may necessitate repeated re-insertion and re-positioning of the flexible tube and associated tools, which takes time away from immediately treating the target tissue and potentially may damage tissue along the pathway.

Certain soft robotic tools have utilized shape memory alloys to manipulate the shape of the tool and facilitate navigation of complex pathways. However, such tools generally may not be ideal for use in surgical applications due to low repeatability, slow response, and relatively high temperatures required for changing from one shape to another through material memory (i.e., transitioning the shape memory alloys from the martensite phase to the austenite phase). Other soft robotic tools have used dielectric actuators for manipulating the shape of the tool. Deformation of dielectric actuators, however, generally may require relatively high voltages that are not suitable for a surgical tool. Still other soft robotic tools have implemented granular jamming mechanisms to manipulate the shape of the tool and provide variable stiffness. However, granular jamming mechanisms generally may be bulky and noisy and may have low force density, making such mechanisms undesirable for use in surgical applications.

A need therefore remains for improved soft robotic tools for navigating complex pathways, such as complex anatomical pathways in MIS applications, which allow the tool to be manipulated to reach a target location simply, quickly, and in one smooth motion.

### SUMMARY OF THE DISCLOSURE

The present disclosure provides soft robotic tools, robotic systems, and related methods for using such tools and systems to navigate complex pathways. In one aspect, a soft robotic tool is provided. In one embodiment, a soft robotic tool may include a plurality of rigid links, a plurality of magnetorheological fluid soft joints, and a plurality of tendons. The rigid links may be disposed in series. Each magnetorheological fluid soft joint may be disposed between a pair of the rigid links. Each magnetorheological fluid soft joint may include a capsule containing a magnetorheological fluid, and an inductive coil disposed around the capsule. The tendons may extend along a length of the soft robotic tool. Each tendon may be attached to each of the rigid links.

In some embodiments, each magnetorheological fluid soft joint may be configured to assume an off state when no magnetic field is generated by the inductive coil and to assume an on state when a magnetic field is generated by the inductive coil. In some embodiments, each magnetorheological fluid soft joint may be configured to allow articulation of the soft robotic tool about the magnetorheological fluid soft joint when the magnetorheological fluid soft joint is in the off state, and each magnetorheological fluid soft joint may be configured to inhibit articulation of the soft robotic tool about the magnetorheological fluid soft joint when the magnetorheological fluid soft joint is in the on state.

In some embodiments, the rigid links may be formed of a polymeric material. In some embodiments, the polymeric material of the rigid links may include acrylonitrile butadiene styrene or polylactic acid. In some embodiments, the rigid links may be formed of a metallic material. In some embodiments, the rigid links may be formed of a ceramic material. In some embodiments, the capsule may be formed of a polymeric material. In some embodiments, the polymeric material of the capsule may include silicone. In some embodiments, the magnetorheological fluid may include a dispersion of magnetic particles in a non-conductive, non-magnetic carrier fluid. In some embodiments, the magnetic particles may include iron particles, and the carrier fluid may include silicone oil. In some embodiments, the magnetorheological fluid also may include one or more surfactants. In some embodiments, the one or more surfactants may include an alkanethiol or a mercaptosilane. In some embodiments, the inductive coil may be encapsulated in a biocompatible polymer. In some embodiments, the tendons may include wires.

In some embodiments, the plurality of tendons may include a first tendon and a second tendon. In some embodiments, the first tendon and the second tendon may extend parallel to one another. In some embodiments, the rigid links may define a first tendon routing pathway and a second tendon routing pathway, with the first tendon extending along the first tendon routing pathway, and with the second tendon extending along the second tendon routing pathway. In some embodiments, the plurality of rigid links may include a first rigid link and a second rigid link, with the first rigid link defining a first portion of the first tendon routing pathway and a first portion of the second tendon routing pathway, and with the second rigid link defining a second portion of the first tendon routing pathway and a second portion of the second tendon routing pathway. In some embodiments, the first portion of the first tendon routing pathway may extend in a linear manner along a length of the first rigid link, and the first portion of the second tendon routing pathway may extend in a linear manner along the length of the first rigid link. In some embodiments, the first portion of the first tendon routing pathway may extend parallel to a longitudinal axis of the first rigid link, and the first portion of the second tendon routing pathway may extend parallel to the longitudinal axis of the first rigid link. In some embodiments, the second portion of the first tendon routing pathway may extend in a linear or non-linear manner along a length of the second rigid link, and the second portion of the second tendon routing pathway may extend in a linear or non-linear manner along the length of the second rigid link. In some embodiments, the second portion of the first tendon routing pathway may be curved along the length of the second rigid link such that a first end of the second portion of the first tendon routing pathway is circumferentially offset from a second end of the second portion of the first tendon routing pathway with respect to a longitudinal axis of the second rigid link, and the second portion of the second tendon routing pathway may be curved along the length of the second rigid link such that a first end of the second portion of the second tendon routing pathway is circumferentially offset from a second end of the second portion of the second tendon routing pathway with respect to the longitudinal axis of the second rigid link. In some embodiments, the first end of the second portion of the first tendon routing pathway may be circumferentially offset from the second end of the second portion of the first tendon routing pathway by 90 degrees, and the first end of the second portion of the second tendon routing pathway may be circumferentially offset from the second end of the second portion of the second tendon routing pathway by 90 degrees. In some embodiments, the plurality of rigid links also may include a third rigid link, with the third rigid link defining a third portion of the first tendon routing pathway and a third portion of the second tendon routing pathway. In some embodiments, the plurality of tendons also includes a third tendon.

In some embodiments, the plurality of magnetorheological fluid soft joints may include a first magnetorheological fluid soft joint and a second magnetorheological fluid soft joint, with the first tendon and the second tendon being configured to bend the first magnetorheological fluid soft joint in a first bending plane, and with the first tendon and the second tendon being configured to bend the second magnetorheological fluid soft joint in a second bending plane transverse to the first bending plane. In some embodiments, the second bending plane may be orthogonal to the first bending plane. In some embodiments, the plurality of rigid links may include a first rigid link, with the first magnetorheological fluid soft joint being connected to a first end of the first rigid link, and with the second magnetorheological fluid soft joint being connected to a second end of the first rigid link. In some embodiments, the plurality of magnetorheological fluid soft joints may include a first magnetorheological fluid soft joint and a second magnetorheological fluid soft joint, with the first tendon and the second tendon being configured to bend the first magnetorheological fluid soft joint in a bending plane, and with the first tendon and the second tendon being configured to bend the second magnetorheological fluid soft joint in the bending plane. In some embodiments, the plurality of magnetorheological fluid soft joints may include a first magnetorheological fluid soft joint, a second magnetorheological fluid soft joint, and a third magnetorheological fluid soft joint. In some embodiments, the plurality of rigid links may include a first rigid link, a second rigid link, and a third rigid link, with each tendon being movably attached to each of the first rigid link and the second rigid link, and with each tendon being fixedly attached to the third rigid link. In some embodiments, the first rigid link may be disposed at a proximal end of the soft robotic tool, the third rigid link may be disposed at a distal end of the soft robotic tool, and the second rigid link may be disposed between the first rigid link and the third rigid link. In some embodiments, each tendon may be movably attached to each of the first rigid link and the second rigid link by passing through respective apertures defined by the first rigid link and the second rigid link.

In another aspect, a soft robotic tool is provided. In one embodiment, a soft robotic tool may include a first rigid link, a second rigid link, a magnetorheological fluid soft joint, a first tendon, and a second tendon. The magnetorheological fluid soft joint may be disposed between the first rigid link and the second rigid link. The magnetorheological fluid soft joint may include a capsule containing a magnetorheological fluid, and an inductive coil disposed around the capsule. The first tendon may be attached to the first rigid link and the second rigid link. The second tendon may be attached to the first rigid link and the second rigid link.

In some embodiments, the magnetorheological fluid soft joint may be configured to assume an off state when no magnetic field is generated by the inductive coil and to assume an on state when a magnetic field is generated by the inductive coil. In some embodiments, the magnetorheological fluid soft joint may be configured to allow articulation of the soft robotic tool about the magnetorheological fluid soft joint when the magnetorheological fluid soft joint is in the off state, and the magnetorheological fluid soft joint may be configured to inhibit articulation of the soft robotic tool about the magnetorheological fluid soft joint when the magnetorheological fluid soft joint is in the on state. In some embodiments, a first end of the magnetorheological fluid soft joint may be connected to the first rigid link, and a second end of the magnetorheological fluid soft joint may be connected to the second rigid link. In some embodiments, the first rigid link may define a first portion of a first tendon routing pathway and a first portion of a second tendon routing pathway, the second rigid link may define a second portion of the first tendon routing pathway and a second portion of the second tendon routing pathway, the first tendon may extend along the first tendon routing pathway, and the second tendon may extend along the second tendon routing pathway. In some embodiments, the first tendon may be movably attached to the first rigid link and fixedly attached to the second rigid link, and the second tendon may be movably attached to the first rigid link and fixedly attached to the second rigid link. In some embodiments, the second rigid link may be disposed at a distal end of the soft robotic tool. In some embodiments, the first tendon may be movably attached to the first rigid link by passing through a first aperture defined by the first rigid link, and the second tendon may be movably attached to the first rigid link by passing through a second aperture defined by the first rigid link.

In still another aspect, a robotic system is provided. In one embodiment, a robotic system may include a soft robotic tool and an actuation module. The soft robotic tool may include a plurality of rigid links, a plurality of magnetorheological fluid soft joints, and a plurality of tendons. The rigid links may be disposed in series. Each magnetorheological fluid soft joint may be disposed between a pair of the rigid links. Each magnetorheological fluid soft joint may include a capsule containing a magnetorheological fluid, and an inductive coil disposed around the capsule. The tendons may extend along a length of the soft robotic tool. Each tendon may be attached to each of the rigid links. The actuation module may include a motor and a plurality of actuators. The motor may be configured to advance and retract the soft robotic tool relative to the actuation module. The actuators may be configured to drive the tendons. Each actuator may be coupled to one of the tendons.

In some embodiments, each magnetorheological fluid soft joint may be configured to assume an off state when no magnetic field is generated by the inductive coil and to assume an on state when a magnetic field is generated by the inductive coil. In some embodiments, each magnetorheological fluid soft joint may be configured to allow articulation of the soft robotic tool about the magnetorheological fluid soft joint when the magnetorheological fluid soft joint is in the off state, and each magnetorheological fluid soft joint may be configured to inhibit articulation of the soft robotic tool about the magnetorheological fluid soft joint when the magnetorheological fluid soft joint is in the on state.

In some embodiments, the actuation module also may include a motor controller configured to control activation of the motor for advancing and retracting the soft robotic tool. In some embodiments, the actuation module also may include an actuator controller configured to control activation of the actuators for driving the tendons to articulate the soft robotic tool about the magnetorheological fluid soft joints. In some embodiments, the actuator controller may be configured to cause only one of the tendons to be pulled while a remainder of the tendons are maintained in a slack state. In some embodiments, the actuation module also includes a plurality of current controllers in communication with the inductive coils of the magnetorheological fluid soft joints, with each current controller being configured to control a strength of a magnetic field generated by one of the inductive coils. In some embodiments, the current controllers may be configured to cause only one of the magnetorheological fluid soft joints to assume the off state while a remainder of the magnetorheological fluid soft joints assume the on state. In some embodiments, the robotic system also may include one or more surgical tools mounted to the soft robotic tool. In some embodiments, the one or more surgical tools may include a camera, a cautery head, or an electrode. In some embodiments, the plurality of rigid links may include a first rigid link, a second rigid link, and a third rigid link, the plurality of tendons may include a first tendon and a second tendon, the first tendon may be movably attached to each of the first rigid link and the second rigid link, the first tendon may be fixedly attached to the third rigid link, the second tendon may be movably attached to each of the first rigid link and the second rigid link, and the second tendon may be fixedly attached to the third rigid link. In some embodiments, the first rigid link may be disposed at a proximal end of the soft robotic tool, the third rigid link may be disposed at a distal end of the soft robotic tool, and the second rigid link may be disposed between the first rigid link and the third rigid link. In some embodiments, the first tendon may be movably attached to each of the first rigid link and the second rigid link by passing through respective apertures defined by the first rigid link and the second rigid link, and the second tendon may be movably attached to each of the first rigid link and the second rigid link by passing through respective apertures defined by the first rigid link and the second rigid link.

In another aspect, a soft robotic tool is provided. In one embodiment, a soft robotic tool may include a plurality of rigid links, and a plurality of magnetorheological fluid soft joints. The rigid links may be disposed in series. Each magnetorheological fluid soft joint may be disposed between a pair of the rigid links. Each magnetorheological fluid soft joint may include a capsule containing a magnetorheological fluid, and an inductive coil disposed around the capsule.

In some embodiments, each magnetorheological fluid soft joint may be configured to assume an off state when no magnetic field is generated by the inductive coil and to assume an on state when a magnetic field is generated by the inductive coil. In some embodiments, each magnetorheological fluid soft joint may be configured to allow articulation of the soft robotic tool about the magnetorheological fluid soft joint when the magnetorheological fluid soft joint is in the off state, and each magnetorheological fluid soft joint may be configured to inhibit articulation of the soft robotic tool about the magnetorheological fluid soft joint when the magnetorheological fluid soft joint is in the on state.

In some embodiments, the rigid links may be formed of a polymeric material. In some embodiments, the polymeric material of the rigid links may include acrylonitrile butadiene styrene or polylactic acid. In some embodiments, the rigid links may be formed of a metallic material. In some embodiments, the rigid links may be formed of a ceramic material. In some embodiments, the capsule may be formed of a polymeric material. In some embodiments, the polymeric material of the capsule may include silicone. In some embodiments, the magnetorheological fluid may include a dispersion of magnetic particles in a non-conductive, non-magnetic carrier fluid. In some embodiments, the magnetic particles may include iron particles, and the carrier fluid may include silicone oil. In some embodiments, the magnetorheological fluid also may include one or more surfactants. In some embodiments, the one or more surfactants may include an alkanethiol or a mercaptosilane. In some embodiments, the inductive coil may be encapsulated in a biocompatible polymer.

These and other aspects and improvements of the present disclosure will become apparent to one of ordinary skill in the art upon review of the following detailed description when taken in conjunction with the several drawings and the appended claims.

### BRIEF DESCRIPTION OF THE DRAWINGS

FIG. 1A schematically illustrates a soft robotic tool in accordance with one or more embodiments of the disclosure, showing three rigid links, a first magnetorheological fluid soft joint, a second magnetorheological fluid soft joint, a first tendon, and a second tendon of the soft robotic tool.

FIG. 1B schematically illustrates the soft robotic tool of FIG. 1A in a first configuration in which the first and second magnetorheological fluid soft joints are in an unlocked state and the first and second tendons are in a slack state.

FIG. 1C schematically illustrates the soft robotic tool of FIG. IA in a second configuration in which the first magnetorheological fluid soft joint is in the unlocked state, the second magnetorheological fluid soft joint is in a locked state, the first tendon is in the slack state, and the second tendon is pulled to articulate the soft robotic tool about the first magnetorheological fluid soft joint.

FIG. 1D schematically illustrates the soft robotic tool of FIG. 1A in a third configuration in which the first magnetorheological fluid soft joint is in the locked state, the second magnetorheological fluid soft joint is in the unlocked state, the second tendon is in the slack state, and the first tendon is pulled to articulate the soft robotic tool about the second magnetorheological fluid soft joint.

FIG. 2A illustrates a perspective view of a soft robotic tool in accordance with one or more embodiments of the disclosure, showing six rigid links, five magnetorheological fluid soft joints, a first tendon, and a second tendon of the soft robotic tool.

FIG. 2B illustrates a detailed perspective view of a portion of the soft robotic tool of FIG. 2A, showing portions of a first tendon routing pathway and a second tendon routing pathway defined by one of the rigid links.

FIG. 2C illustrates a detailed perspective view of another portion of the soft robotic tool of FIG. 2A, showing portions of the first tendon routing pathway and the second tendon routing pathway defined by another one of the rigid links.

FIG. 3 schematically illustrates a robotic system in accordance with one or more embodiments of the disclosure, showing a robotic tool and an actuation module of the robotic system.

The detailed description is set forth with reference to the accompanying drawings. The drawings are provided for purposes of illustration only and merely depict example embodiments of the disclosure. The drawings are provided to facilitate understanding of the disclosure and shall not be deemed to limit the breadth, scope, or applicability of the disclosure. The use of the same reference numerals indicates similar, but not necessarily the same or identical components. Different reference numerals may be used to identify similar components. Various embodiments may utilize elements or components other than those illustrated in the drawings, and some elements and/or components may not be present in various embodiments. The use of singular terminology to describe a component or element may, depending on the context, encompass a plural number of such components or elements and vice versa.

### DETAILED DESCRIPTION OF THE DISCLOSURE

In the following description, specific details are set forth describing some embodiments consistent with the present disclosure. Numerous specific details are set forth in order to provide a thorough understanding of the embodiments. It will be apparent, however, to one skilled in the art that some embodiments may be practiced without some or all of these specific details. The specific embodiments disclosed herein are meant to be illustrative but not limiting. One skilled in the art may realize other elements that, although not specifically described here, are within the scope and the spirit of this disclosure. In addition, to avoid unnecessary repetition, one or more features shown and described in association with one embodiment may be incorporated into other embodiments unless specifically described otherwise or if the one or more features would make an embodiment non-functional. In some instances, well known methods, procedures, components, and circuits have not been described in detail so as not to unnecessarily obscure aspects of the embodiments.

Embodiments of soft robotic tools, robotic systems, and related methods for using such tools and systems to navigate complex pathways are provided. As described herein, the soft robotic tools advantageously may be well suited for navigating complex anatomical pathways in MIS applications. In one embodiment, a soft robotic tool may include a plurality of rigid links disposed in series, a plurality of magnetorheological fluid soft joints each disposed between a pair of the rigid links, and a plurality of tendons each attached to each of the rigid links. Each magnetorheological fluid soft joint may include a capsule containing a magnetorheological fluid, and an inductive coil disposed around the capsule. When no magnetic field is generated by the inductive coil, the magnetorheological fluid soft joint may assume an unlocked or off state in which the soft robotic tool may be articulated about the magnetorheological fluid soft joint. For example, one of the tendons may be pulled to bend the magnetorheological fluid soft joint in a predefined bending plane. When a magnetic field is generated by the inductive coil, the magnetorheological fluid soft joint may assume a locked or on state in which the soft robotic tool is inhibited from articulating about the magnetorheological fluid soft joint. During use of the soft robotic tool, one of the magnetorheological fluid soft joints may be unlocked while a remainder of the magnetorheological fluid soft joints are locked, such that one of the tendons may be pulled to articulate the soft robotic tool about the unlocked magnetorheological fluid soft joint. The magnetorheological fluid soft joints may be sequentially unlocked and locked, and the tendons may be selectively pulled to bend, aim, and orient the soft robotic tool as desired to facilitate navigation of complex pathways. In this manner, the magnetorheological fluid soft joints may function as an embedded switching mechanism, with the magnetorheological fluid controlling mobility of the magnetorheological fluid soft joints, while the tendons control motion actuation of the soft robotic tool. Ultimately, the soft robotic tool described herein may overcome the above-described limitations associated with use of existing soft robotic tools to navigate complex anatomical pathways. In particular, the magnetorheological fluid soft joints may provide a compact, responsive means for enabling and disabling mobility of the respective joints, while the tendons provide an accurate, repeatable means for precisely controlling motion actuation of the soft robotic tool.

Although the soft robotic tools, robotic systems, and related methods provided herein may be described as being particularly useful for surgical applications, it will be appreciated that the use of such tools, systems, and methods is not limited to surgical applications. To the contrary, the soft robotic tools, robotic systems, and related methods described herein advantageously may be used in various non-surgical and non-medical applications in which navigation of complex pathways including non-linear portions, multiple branches, and/or changes in diameter, such as changes from relatively large diameters to relatively small diameters, is desirable.

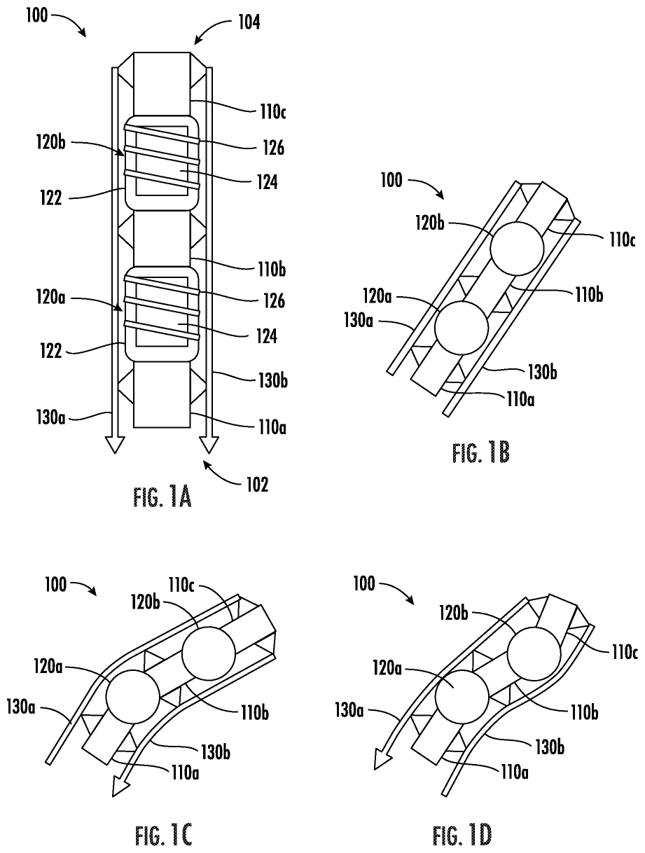

Referring now to FIG. 1A, a soft robotic tool 100 (also referred to herein as a “robotic surgical tool,” a “robotic tool” or simply a “tool”) in accordance with one or more embodiments of the disclosure is depicted. The soft robotic tool 100 is configured for navigating complex pathways. For example, the soft robotic tool 100 may be used for navigating complex anatomical pathways, such as in MIS applications. The soft robotic tool 100 may be formed as an elongated structure having a proximal end 102 and a distal end 104 disposed opposite one another along a longitudinal axis of the tool 100. Although the soft robotic tool 100 is depicted in a linear configuration in FIG. 1A, the tool 100 may be articulated from the linear configuration into various non-linear configurations, as described below with respect to FIGS. 1B-1D. As shown in FIG. 1A, the soft robotic tool 100 may include a plurality of rigid links 110, a plurality of magnetorheological fluid soft joints 120, and a plurality of tendons 130.

The rigid links 110 (also referred to herein as “links”) may be disposed in series along the length of the soft robotic tool 100. In some embodiments, as shown, the rigid links 110 may include a first rigid link 110a, a second rigid link 110b, and a third rigid link 110c. In other embodiments, any number of the rigid links 110 may be used, such as four or more rigid links 110, depending on the intended use and desired length of the soft robotic tool 100. The rigid links 110 may be formed as rigid members that do not deform, elastically or plastically, during use of the soft robotic tool 100 for its intended purpose. In some embodiments, the rigid links 110 may be formed of a polymeric material, a metallic material, or a ceramic material, although other suitable biocompatible materials may be used for the links 110. In some embodiments the polymeric material of the rigid links 110 may include acrylonitrile butadiene styrene (ABS) or polylactic acid (PIA), although other suitable biocompatible polymeric materials may be used for the links 110. The rigid links 110 may have various regular or irregular shapes. In some embodiments, the rigid links 110 each may have a cylindrical shape with a circular cross-sectional shape, although other suitable shapes may be used for the links 110. The rigid links 110 each may have a length in the direction of the longitudinal axis of the soft robotic tool 100 and a width (i.e., a diameter when the links 110 have a circular cross-sectional shape) in the direction orthogonal to the longitudinal axis of the tool 100. In some embodiments, for each of the rigid links 110, the length of the link 110 may be greater than the width of the link 110 In other embodiments, for each of the rigid links 110, the length of the link 110 may be less than or equal to the width of the link 110. In some embodiments, all of the rigid links 110 may have the same shape and dimensions. In other embodiments, one or more of the rigid links 110 may have a shape and/or dimension that is different from the shape and/or dimension of one or more of the other rigid links 110.

The magnetorheological fluid soft joints 120 (also referred to herein as “magnetorheological fluidic joints,” “magnetorheological joints,” or simply “joints”) may be disposed in series along the length of the soft robotic tool 100 and interspersed among the rigid links 110. As shown, each magnetorheological fluid soft joint 120 may be disposed between and connected to a consecutive pair of the rigid links 110. In some embodiments, as shown, the magnetorheological fluid soft joints 120 may include a first magnetorheological fluid soft joint 120a and a second magnetorheological fluid soft joint 120b. The first magnetorheological fluid soft joint 120a may be connected to a distal end of the first rigid link 110a and a proximal end of the second rigid link 110b. The second magnetorheological fluid soft joint 120b may be connected to a distal end of the second rigid link 110b and a proximal end of the third rigid link 110c. In other embodiments, any number of the magnetorheological fluid soft joints 120 may be used, such as three or more magnetorheological fluid soft joints 120, depending on the intended use and desired length of the soft robotic tool 100.

As shown, each magnetorheological fluid soft joint 120 may include a capsule 122 containing a magnetorheological fluid 124 therein, and an inductive coil 126 disposed around the capsule 122. The capsule 122 may be formed as a flexible container that allows the magnetorheological fluid soft joint 120 to bend in a bending plane when the joint 120 is in an unlocked or off state, as described below. In some embodiments, the capsule 122 may be formed of a polymeric material, although other suitable biocompatible materials may be used for the capsule 122. In some embodiments the polymeric material of the capsule 122 may include silicone, although other suitable biocompatible polymeric materials may be used for the capsule 122. The capsules 122 may have various regular or irregular shapes when the capsule 122 is in a natural state (i.e., absent external forces acting on the capsule 122), although the capsule 122 may be elastically deformed to various other shapes during use of the soft robotic tool 100. In some embodiments, the capsules 122 each may have a cylindrical shape with a circular cross-sectional shape, although other suitable shapes may be used for the capsules 122. The capsules 122. each may have a length in the direction of the longitudinal axis of the soft robotic tool 100 and a width (i.e., a diameter when the capsules 122 have a circular cross-sectional shape) in the direction orthogonal to the longitudinal axis of the tool 100. In some embodiments, for each of the capsules 122, the length of the capsule 122 may be greater than the width of the capsule 122. In other embodiments, for each of the capsules 122, the length of the capsule 122 may be less than or equal to the width of the capsule 122. In some embodiments, all of the capsules 122 may have the same shape and dimensions. In other embodiments, one or more of the capsules 122 may have a shape and/or dimension that is different from the shape and/or dimension of one or more of the other capsules 122. In some embodiments, the length of the capsules 122 may be greater than the length of the rigid links 110. In other embodiments, the length of the capsules 122 may be less than or equal to the length of the rigid links 110.

For each magnetorheological fluid soft joint 120, the magnetorheological fluid 124 may include a dispersion of magnetic particles in a non-conductive, non-magnetic carrier fluid. In some embodiments, the magnetic particles may include iron particles, although other suitable magnetic particles may be used for the magnetorheological fluid 124. In some embodiments, the carrier fluid may include silicone oil or mineral oil, although other suitable carrier fluids may be used for the magnetorheological fluid 124. The magnetorheological fluid 124 may be configured to transition between an on or magnetized state and an off or un-magnetized state, based on the application of a magnetic field or absence of a magnetic field. When in the off state, the magnetorheological fluid 124 may exhibit similar fluid behavior to the carrier fluid, which may be generally similar in viscosity to the carrier fluid and Newtonian or slightly shear thinning. When in the on state, the magnetic particles in the magnetorheological fluid 124 may align with the magnetic field and form chains, with such alignment restricting bulk fluid flow and typically changing the fluid rheological properties to that of a Bingham plastic. In this manner, the magnetorheological fluid 124 may show a critical yield stress rather than a continuous relationship between stress and strain. For the magnetorheological fluid 124, the magnetized viscosity below the yield stress may be significantly higher than that of the non-magnetized fluid. Above the yield stress, the viscosity of the magnetorheological fluid 124 may be significantly lower and may be expected to be Newtonian or shear thinning. The magnetorheological fluid 124 may be optimized to have a low non-magnetized viscosity and a high yield stress by adjusting the formulation of the magnetorheological fluid 124 and/or varying the magnetic field applied during use of the soft robotic tool 100. Variables in determining the formulation of the magnetorheological fluid 124 include the chemistry and viscosity of the carrier fluid, the chemistry, shape, size, and concentration of the magnetic particles, and the addition of additives, if any. In some embodiments, the magnetorheological fluid 124 may include one or more surfactants, for example, for mitigating potential settling of the magnetic particles. In some embodiments, the one or more surfactants may include an alkanethiol or a mercaptosilane.

For each magnetorheological fluid soft joint 120, the inductive coil 126 may be disposed around the capsule 122. The inductive coil 126 may include a wire formed of a conductive, metallic material. In some embodiments, the inductive coil 126 may be encapsulated in a thin layer of biocompatible polymer for inhibiting any negative interactions with biological fluids and resisting corrosion. As shown, the inductive coil 126 may be wound around the capsule 122. Although the inductive coil 126 illustrated in FIG. 1A includes three turns, the inductive coil 126 may include any number of turns as needed to provide a desired strength of the magnetic field without substantially altering the stiffness of the capsule 122.

The tendons 130 (also referred to herein as “driving tendons” or “wires”) may extend along the length of the soft robotic tool 100. In some embodiments, as shown, the tendons 130 may include a first tendon 130a and a second tendon 130b each extending along the length of soft robotic tool 100. In some embodiments, the first tendon 130a and the second tendon 130b may extend parallel to one another along at least a portion of the length of the soft robotic tool 100. For example, the first tendon 130a and the second tendon 130b may extend parallel to one another along at least the distalmost magnetorheological fluid soft joint 120. In some embodiments, the first tendon 130a and the second tendon 130b may extend along opposite sides of the soft robotic tool 100. For example, the first tendon 130a and the second tendon 130b may be circumferentially spaced apart from one another by 180 degrees with respect to the longitudinal axis of the tool 100. Alternatively, the first tendon 130a and the second tendon 130b may be spaced apart from one another by a circumferential offset other than 180 degrees. In some embodiments, more than two tendons 130 may be used. For example, three, four, five, six, seven, eight, or more tendons 130 may be used, with the tendons 130 being equally or unequally spaced apart from one another in a circumferential array with respect to the longitudinal axis of the tool 100. In some embodiments, the number of tendons 130 used may depend on the overall diameter of the tool 100. As shown, each tendon 130 may be attached to each of the rigid links 110. In some embodiments, each tendon 130 may be fixedly attached to the distalmost rigid link 110 and movably attached to the remainder of the rigid links 110. For example, according to the embodiment illustrated in FIG. 1A, the first tendon 130a and the second tendon 130b each may be fixedly attached to the third rigid link 110c, and the first tendon 130a and the second tendon 130b each may be movably attached to each of the first rigid link 110a and the second rigid link 110b. In some embodiments, the tendons 130 may be fixedly attached to the distalmost rigid link 110 by adhesive, fasteners, or other means for mechanically fixing the tendons 130 to the distalmost rigid link 110. In some embodiments, the tendons 130 may be attached to the remainder of the rigid links 110 by passing through respective portions of tendon routing pathways defined by the rigid links 110. For example, each rigid link 110 may define a portion of a first tendon routing pathway for the first tendon 130a, and each rigid link 110 may define a portion of a second tendon routing pathway for the second tendon 130b. The respective portions of the tendon routing pathways may be formed as apertures, channels, or other suitable features for receiving a portion of the respective tendon 130 therethrough. Other suitable means and configurations for attaching the tendons 130 to the rigid links 110 may be used in other embodiments. As shown, each tendon 130 may be unattached with respect to the magnetorheological fluid soft joints 120. The tendons 130 may be formed as elongated, flexible members for controlling motion actuation of the soft robotic tool 100. In some embodiments, each tendon 130 may include a single wire or a plurality of wires. In some embodiments, the tendons 130 may be formed of a polymeric material, although other suitable biocompatible materials may be used for the tendons 130.

FIGS. 1B-1D illustrate an example of how the soil robotic tool 100 may be articulated during use of the tool 100, for example in navigating a complex pathway. FIG. 1B shows the soft robotic tool 100 in a first configuration in which the tool 100 has a linear shape. In the first configuration, the first and second magnetorheological fluid soft joints 120a, 120b are in an unlocked or off state (i.e., the magnetorheological fluid 124 is in the off state due to an absence of a magnetic field applied by the inductive coils 126 of the joints 120a, 120b), and the first and second tendons 130a, 130b are in a slack state. As used herein with respect to a tendon, the term “slack state” refers to a state in which the tendon is not being pulled taut. Further, in the first configuration, the longitudinal axes of the rigid links 110a, 110b, 110c are coaxial with one another. The first configuration may be used when the soft robotic tool 100 is advanced along a linear portion of the complex pathway.

As the distal end 104 of the soft surgical tool 100 approaches a first non-linear or branched portion of the complex pathway, the tool 100 may be moved to a second configuration in which the tool 100 has a non-linear shape, as shown in FIG. 1C. To transition from the first configuration to the second configuration, the second magnetorheological fluid soft joint 120b may be switched from the unlocked state to a locked or on state (i.e., the magnetorheological fluid 124 is switched from the off state to the on state due to a magnetic field applied by the inductive coil 126 of the second joint 120b), and the second tendon 130b may be pulled proximally, while the first magnetorheological fluid soft joint 120a is maintained in the unlocked state and the first tendon 130a is maintained in the slack state. As a result, the pulling of the second tendon 130b may cause the first magnetorheological fluid soft joint 120a to bend while the second magnetorheological fluid soft joint 120b remains locked. In this manner, the bending of the first magnetorheological fluid soft joint 120a may cause the longitudinal axes of the first and second rigid links 110a, 110b to be angled with respect to one another, while the longitudinal axes of the second and third rigid links 110b, 110c remain coaxial with one another.

As the distal end 104 of the soft surgical tool 100 approaches a second non-linear or branched portion of the complex pathway extending in a direction different from the first non-linear or branched portion, the tool 100 may be moved to a third configuration in which the tool 100 has a different non-linear shape, as shown in FIG. 1D. To transition from the second configuration to the third configuration, the first magnetorheological fluid soft joint 120a may be switched from the unlocked state to the locked state (i.e., the magnetorheological fluid 124 is switched from the off state to the on state due to a magnetic field applied by the inductive coil 126 of the first joint 120a), the second magnetorheological fluid soft joint 120b may be switched from the locked state to the unlocked state (i.e., the magnetorheological fluid 124 is switched. from the on state to the off state due to an absence of a magnetic field applied by the inductive coil 126 of the second joint 120b), and the first tendon 130a may be pulled proximally, while the second tendon 130b is transitioned to the slack state. As a result, the pulling of the first tendon 130a may cause the second magnetorheological fluid soft joint 120b to bend while the first magnetorheological fluid soft joint 120a remains locked. In this manner, the bending of the second magnetorheological fluid soft joint 120b may cause the longitudinal axes of the second and third rigid links 110b, 110c to be angled with respect to one another, while the existing angle between the longitudinal axes of the first and second rigid links 110a, 110b is maintained.

It will be appreciated that the configurations of the soft robotic tool 100 shown in FIGS. 1B-1D are merely a few examples of how the tool 100 may be articulated. For example, the degree of angulation between consecutive rigid links 110 provided by bending of the magnetorheological fluid soft joints 120 may be varied, as needed, to navigate a complex pathway. Further, although the illustrated example shows the magnetorheological fluid soft joints 120 bending in a common bending plane, in other embodiments, the joints may bend in different bending planes that are transverse to one another, as described below. Additionally, as the soft robotic tool 100 is advanced along a complex pathway, the entire tool 100 may be rotated within the pathway to facilitate navigation in multiple planes. Further, as described above, the soft robotic tool 100 may include more than two magnetorheological fluid soft joints 120 to provide additional degrees of freedom for articulating the tool 100. Finally, the soft robotic tool 100 may include more than two tendons 130 to facilitate articulation of the tool 100 in additional bending planes.

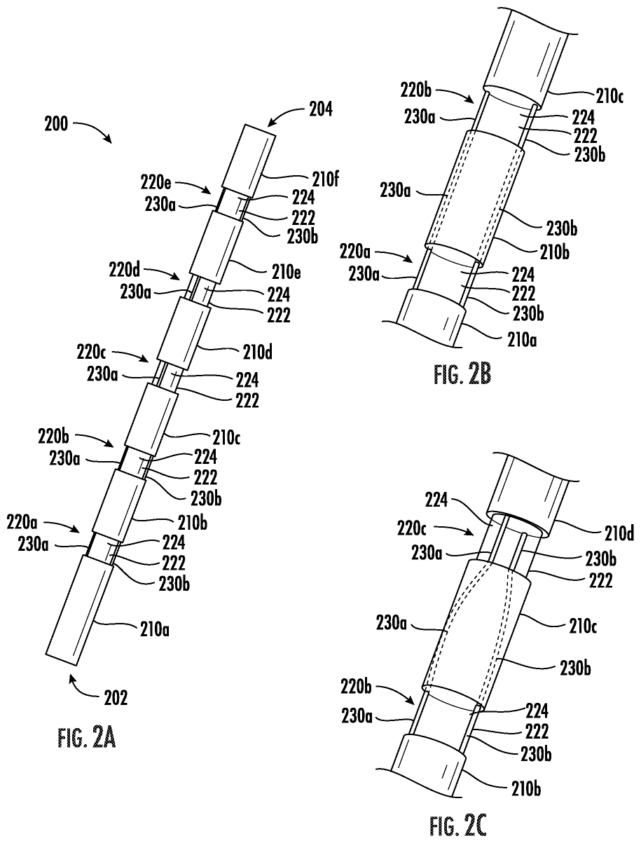

FIGS. 2A-2C depict a soft robotic tool 200 (also referred to herein as a “robotic surgical tool,” a “robotic tool” or simply a “tool”) in accordance with one or more embodiments of the disclosure. It will be appreciated that the soft robotic tool 200 generally may be configured in a manner similar to the soft robotic tool 100, although certain differences are described herein. The soft robotic tool 200 is configured for navigating complex pathways. For example, the soft robotic tool 200 may be used for navigating complex anatomical pathways, such as in MIS applications. The soft robotic tool 200 may be formed as an elongated structure having a proximal end 202 and a distal end 204 disposed opposite one another along a longitudinal axis of the tool 200. Although the soft robotic tool 200 is depicted in a linear configuration in FIG. 2A, the tool 200 may be articulated from the linear configuration into various non-linear configurations, as described below. As shown in FIG. 2A, the soft robotic tool 200 may include a plurality of rigid links 210, a plurality of magnetorheological fluid soft joints 220, and a plurality of tendons 230.

The rigid links 210 (also referred to herein as “links”) may be disposed in series along the length of the soft robotic tool 200. In some embodiments, as shown, the rigid links 210 may include a first rigid link 210a, a second rigid link 210b, a third rigid link 210c, a fourth rigid link 210d, a fifth rigid link 210e, and a sixth rigid link 210f. In other embodiments, any number of the rigid links 210 may be used, such as seven or more rigid links 210, depending on the intended use and desired length of the soft robotic tool 200. The rigid links 210 may be formed as rigid members that do not deform, elastically or plastically, during use of the soft robotic tool 200 for its intended purpose. In some embodiments, the rigid links 210 may be formed of a polymeric material, a metallic material, or a ceramic material, although other suitable biocompatible materials may be used for the links 210. In some embodiments the polymeric material of the rigid links 210 may include acrylonitrile butadiene styrene (ABS) or polylactic acid (PLA), although other suitable biocompatible polymeric materials may be used for the links 210. The rigid links 210 may have various regular or irregular shapes. In some embodiments, the rigid links 210 each may have a cylindrical shape with a circular cross-sectional shape, although other suitable shapes may be used for the links 210. The rigid links 210 each may have a length in the direction of the longitudinal axis of the soft robotic tool 200 and a width (i.e., a diameter when the links 210 have a circular cross-sectional shape) in the direction orthogonal to the longitudinal axis of the tool 200. In some embodiments, for each of the rigid links 210, the length of the link 210 may be greater than the width of the link 210 In other embodiments, for each of the rigid links 210, the length of the link 210 may be less than or equal to the width of the link 210. In some embodiments, all of the rigid links 210 may have the same shape and dimensions. In other embodiments, one or more of the rigid links 210 may have a shape and/or dimension that is different from the shape and/or dimension of one or more of the other rigid links 210.

The magnetorheological fluid soft joints 220 (also referred to herein as “magnetorheological fluidic joints,” “magnetorheological joints,” or simply “joints”) may be disposed in series along the length of the soft robotic tool 200 and interspersed among the rigid links 210. As shown, each magnetorheological fluid soft joint 220 may be disposed between and connected to a consecutive pair of the rigid links 210. In some embodiments, as shown, the magnetorheological fluid soft joints 220 may include a first magnetorheological fluid soft joint 220a, a second magnetorheological fluid soft joint 220b, a third magnetorheological fluid soft joint 220c, a fourth magnetorheological fluid soft joint 220d, and a fifth magnetorheological fluid soft joint 220e. The first magnetorheological fluid soft joint 220a may be connected to a distal end of the first rigid link 210a and a proximal end of the second rigid link 210b. The second magnetorheological fluid soft joint 220b may be connected to a distal end of the second rigid link 210b and a proximal end of the third rigid link 210c. The third magnetorheological fluid soft joint 220c may be connected to a distal end of the third rigid link 210c and a proximal end of the fourth rigid link 210d. The fourth magnetorheological fluid soft joint 220d may be connected to a distal end of the fourth rigid link 210d and a proximal end of the fifth rigid link 210e. The fifth magnetorheological fluid soft joint 220e may be connected to a distal end of the fifth rigid link 210e and a proximal end of the sixth rigid link 210f. In other embodiments, any number of the magnetorheological fluid soft joints 220 may be used, such as six or more magnetorheological fluid soft joints 220, depending on the intended use and desired length of the soft robotic tool 200.

As shown, each magnetorheological fluid soft joint 220 may include a capsule 222 containing a magnetorheological fluid 224 therein. Each magnetorheological fluid soft joint 220 also may include an inductive coil (not shown in FIGS. 2A-2C) disposed around the capsule 222. The capsule 222 may be formed as a flexible container that allows the magnetorheological fluid soft joint 220 to bend in a bending plane when the joint 220 is in an unlocked or off state, as described below. In some embodiments, the capsule 222 may be formed of a polymeric material, although other suitable biocompatible materials may be used for the capsule 222. In some embodiments the polymeric material of the capsule 222 may include silicone, although other suitable biocompatible polymeric materials may be used for the capsule 222. The capsules 222 may have various regular or irregular shapes when the capsule 222 is in a natural state (i.e., absent external forces acting on the capsule 222), although the capsule 222 may be elastically deformed to various other shapes during use of the soft robotic tool 200. In some embodiments, the capsules 222 each may have a cylindrical shape with a circular cross-sectional shape, although other suitable shapes may be used for the capsules 222. The capsules 222 each may have a length in the direction of the longitudinal axis of the soft robotic tool 200 and a width (i.e., a diameter when the capsules 222 have a circular cross-sectional shape) in the direction orthogonal to the longitudinal axis of the tool 200. In some embodiments, for each of the capsules 222, the length of the capsule 222 may be greater than the width of the capsule 222. In other embodiments, for each of the capsules 222, the length of the capsule 222 may be less than or equal to the width of the capsule 222. In some embodiments, all of the capsules 222 may have the same shape and dimensions. In other embodiments, one or more of the capsules 222 may have a shape and/or dimension that is different from the shape and/or dimension of one or more of the other capsules 222. In some embodiments, the length of the capsules 222 may be greater than the length of the rigid links 210. In other embodiments, the length of the capsules 222 may be less than or equal to the length of the rigid links 210.

For each magnetorheological fluid soft joint 220, the magnetorheological fluid 224 may include a dispersion of magnetic particles in a non-conductive, non-magnetic carrier fluid. In some embodiments, the magnetic particles may include iron particles, although other suitable magnetic particles may be used for the magnetorheological fluid 224. In some embodiments, the carrier fluid may include silicone oil or mineral oil, although other suitable carrier fluids may be used for the magnetorheological fluid 224. The magnetorheological fluid 224 may be configured to transition between an on or magnetized state and an off or un-magnetized state, based on the application of a magnetic field or absence of a magnetic field. When in the off state, the magnetorheological fluid 224 may exhibit similar fluid behavior to the carrier fluid, which may be generally similar in viscosity to the carrier fluid and Newtonian or slightly shear thinning. When in the on state, the magnetic particles in the magnetorheological fluid 224 may align with the magnetic field and form chains, with such alignment restricting bulk fluid flow and typically changing the fluid rheological properties to that of a Bingham plastic. In this manner, the magnetorheological fluid 224 may show a critical yield stress rather than a continuous relationship between stress and strain. For the magnetorheological fluid 224, the magnetized viscosity below the yield stress may be significantly higher than that of the non-magnetized fluid. Above the yield stress, the viscosity of the magnetorheological fluid 224 may be significantly lower and may be expected to be Newtonian or shear thinning. The magnetorheological fluid 224 may be optimized to have a low non-magnetized viscosity and a high yield stress by adjusting the formulation of the magnetorheological fluid 224 and/or varying the magnetic field applied during use of the soft robotic tool 200. Variables in determining the formulation of the magnetorheological fluid 224 include the chemistry and viscosity of the carrier fluid, the chemistry, shape, size, and concentration of the magnetic particles, and the addition of additives, if any. In some embodiments, the magnetorheological fluid 224 may include one or more surfactants, for example, for mitigating potential settling of the magnetic particles. In some embodiments, the one or more surfactants may include an alkanethiol or a mercaptosilane.

For each magnetorheological fluid soft joint 220, the inductive coil may be disposed around the capsule 222. The inductive coil may include a wire formed of a conductive, metallic material. In some embodiments, the inductive coil may be encapsulated in a thin layer of biocompatible polymer for inhibiting any negative interactions with biological fluids and resisting corrosion. The inductive coil may be configured in a manner similar to the inductive coil 126 described above and shown in FIG. 1A, with the inductive coil wound around the capsule 222. The inductive coil may include any number of turns as needed to provide a desired strength of the magnetic field without substantially altering the stiffness of the capsule 222.

The tendons 230 (also referred to herein as “driving tendons” or “wires”) may extend along the length of the soft robotic tool 200. In some embodiments, as shown, the tendons 230 may include a first tendon 230a and a second tendon 230b each extending along the length of soft robotic tool 200. In some embodiments, the first tendon 230a and the second tendon 230b may extend parallel to one another along at least a portion of the length of the soft robotic tool 200. For example, the first tendon 230a and the second tendon 230b may extend parallel to one another along at least the distalmost magnetorheological fluid soft joint 220. In some embodiments, the first tendon 230a and the second tendon 230b may extend along opposite sides of the soft robotic tool 200. For example, the first tendon 230a and the second tendon 230b may be circumferentially spaced apart from one another by 180 degrees with respect to the longitudinal axis of the tool 200 over at least a portion of the length of the tool 200. Alternatively, the first tendon 230a and the second tendon 230b may be spaced apart from one another by a circumferential offset other than 180 degrees. In some embodiments, more than two tendons 230 may be used. For example, three, four, five, six, seven, eight, or more tendons 230 may be used, with the tendons 230 being equally or unequally spaced apart from one another in a circumferential array with respect to the longitudinal axis of the tool 200 over at least a portion of the length of the tool 200. In some embodiments, the number of tendons 230 used may depend on the overall diameter of the tool 200. As shown, each tendon 230 may be attached to each of the rigid links 210. In some embodiments, each tendon 230 may be fixedly attached to the distalmost rigid link 210 and movably attached to the remainder of the rigid links 210. For example, according to the embodiment illustrated in FIGS. 2A-2C, the first tendon 230a and the second tendon 230b each may be fixedly attached to the sixth rigid link 210f, and the first tendon 230a and the second tendon 230b each may be movably attached to each of the first rigid link 210a, the second rigid link 210b, the third rigid link 210c, the fourth rigid link 210d, and the fifth rigid link 210e. In some embodiments, the tendons 230 may be fixedly attached to the distalmost rigid link 210 by adhesive, fasteners, or other means for mechanically fixing the tendons 230 to the distalmost rigid link 210. In some embodiments, the tendons 230 may be attached to the remainder of the rigid links 210 by passing through respective portions of tendon routing pathways defined by the rigid links 210. For example, in the illustrated embodiment, each rigid link 210 may define a portion of a first tendon routing pathway for the first tendon 230a, and each rigid link 210 may define a portion of a second tendon routing pathway for the second tendon 230b. The respective portions of the tendon routing pathways may be formed as apertures, channels, or other suitable features for receiving a portion of the respective tendon 230 therethrough. Other suitable means and configurations for attaching the tendons 230 to the rigid links 210 may be used in other embodiments. As shown, each tendon 230 may be unattached with respect to the magnetorheological fluid soft joints 220. The tendons 230 may be formed as elongated, flexible members for controlling motion actuation of the soft robotic tool 200. In some embodiments, each tendon 230 may include a single wire or a plurality of wires. In some embodiments, the tendons 230 may be formed of a polymeric material, although other suitable biocompatible materials may be used for the tendons 230.

FIGS. 2B and 2C provide detailed views showing example portions of the first tendon routing pathway and the second tendon routing pathway defined by the rigid links 210. FIG. 2B shows the second rigid link 210b and the portions of the first tendon routing pathway and the second tendon routing pathway defined by the second rigid link 210b, with the first tendon 230a and the second tendon 230b extending along the respective portions of the tendon routing pathways. As shown, the portion of the first tendon routing pathway defined by the second rigid link 210b may extend in a linear manner along the length of the second rigid link 210b, and the portion of the second tendon routing pathway defined by the second rigid link 210b may extend in a linear manner along the length of the second rigid link 210b. In some embodiments, as shown, the portion of the first tendon routing pathway defined by the second rigid link 210b and the portion of the second tendon routing pathway defined by the second rigid link 210b each may extend parallel to the longitudinal axis of the second rigid link 210b. In this manner, along the length of the second rigid link 210b, the first tendon 230a and the second tendon 230b each may extend in a linear manner and parallel to the longitudinal axis of the second rigid link 210b.

FIG. 2C shows the third rigid link 210c and the portions of the first tendon routing pathway and the second tendon routing pathway defined by the third rigid link 210c, with the first tendon 230a and the second tendon 230b extending along the respective portions of the tendon routing pathways. As shown, the portion of the first tendon routing pathway defined by the third rigid link 210c may extend in a non-linear manner along the length of the third rigid link 210c, and the portion of the second tendon routing pathway defined by the third rigid link 210c may extend in a non-linear manner along the length of the third rigid link 210c. In some embodiments, as shown, the portion of the first tendon routing pathway defined by the third rigid link 210c may be curved along the length of the third rigid link 210c such that a proximal end of the portion of the first tendon routing pathway is circumferentially offset from a distal end of the portion of the first tendon routing pathway with respect to the longitudinal axis of the third rigid link 210c. For example, the proximal end and the distal end of the portion of the first tendon routing pathway defined by the third rigid link 210c may be circumferentially offset from one another by 90 degrees, as shown. Similarly, the portion of the second tendon routing pathway defined by the third rigid link 210c may be curved along the length of the third rigid link 210c such that a proximal end of the portion of the second tendon routing pathway is circumferentially offset from a distal end of the portion of the second tendon routing pathway with respect to the longitudinal axis of the third rigid link 210c. For example, the proximal end and the distal end of the portion of the second tendon routing pathway defined by the third rigid link 210c may be circumferentially offset from one another by 90 degrees, as shown.

The configurations of the first tendon routing pathway and the second tendon routing pathway shown in FIGS. 2A-2C may facilitate bending of different magnetorheological fluid soft joints 220 in different bending planes or the same bending plane. According to the illustrated configurations, the tendons 230a, 230b may be configured to bend the first magnetorheological fluid soft joint 220a, the second magnetorheological fluid soft joint 220b, and the fifth magnetorheological fluid soft joint 220e in a first bending plane, and the tendons 230a, 230b may be configured to bend the third magnetorheological fluid soft joint 220c and the fourth magnetorheological fluid soft joint 220d in a second bending plane that is transverse to the first bending plane. For example, the second bending plane may be orthogonal to the first bending plane, although other transverse orientations may be used. In this manner, the tendon routing pathways may allow the tendons 230 to bend the magnetorheological fluid soft joints 220 in multiple bending planes to achieve various articulated configurations of the soft robotic tool 200. It will be appreciated that the configurations of tendon routing pathways of the soft robotic tool 200 shown in FIGS. 2A-2C are merely one example, and that other configurations may be used for bending the magnetorheological fluid soft joints 220 in two, three, or more different bending plans.

During use, the soft robotic tool 200 may be articulated in various configurations, in a manner similar to the soft robotic tool 100 described above with reference to FIGS. 1B-1D, for navigating a complex pathway. In particular, the magnetorheological fluid soft joints 220 may be sequentially switched from a locked or on state (i.e., in which the magnetorheological fluid 224 is in the on state due to magnetic fields applied by the inductive coils of the joints 220) to an unlocked or off state (i.e., in which the magnetorheological fluid 224 is in the off state due to an absence of a magnetic field applied by the inductive coils of the joints 220), and one of the tendons 230 may be pulled while the other tendon 230 is in a slack state to bend the unlocked magnetorheological fluid soft joint 220. In this manner, the soft robotic tool 200 may be transitioned from one configuration to another to facilitate navigation of the complex pathway.

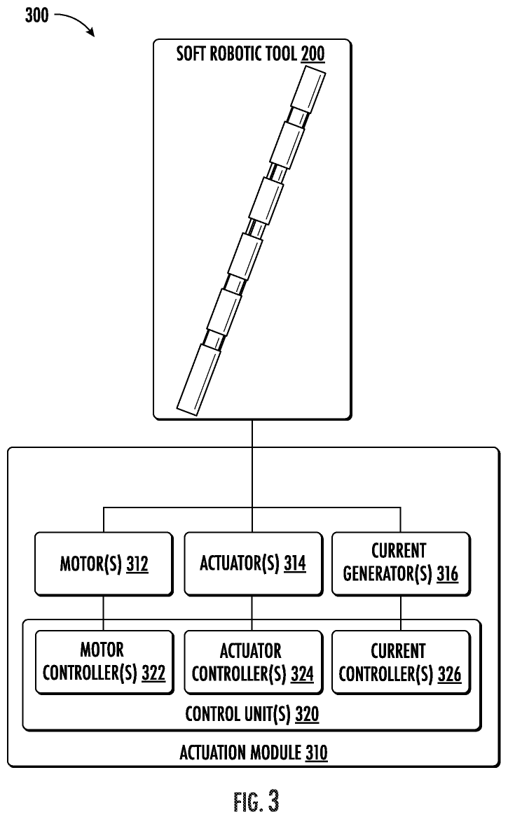

FIG. 3 depicts a robotic system 300 (also referred to herein as a “soft robotic system,” a “robot,” or simply a “system”) in accordance with one or more embodiments of the disclosure. The robotic system 300 may be configured for navigating complex pathways and performing a procedure. For example, the robotic system 300 may be configured for navigating anatomical complex pathways and performing a surgical procedure, such as an MIS procedure. As shown in FIG. 3, the robotic system 300 may include the soft robotic tool 200 described above and an actuation module 310 configured to guide and articulate the soft robotic tool 200 for navigating a complex pathway and performing a procedure.

As shown, the actuation module 310 may include one or more motor(s) 312, one or more actuator(s) 314, one or more current generator(s) 316, and one or more control unit(s) 320. The motor(s) 312 may be configured to advance and retract the soft robotic tool 200 relative to the actuation module 310. In this manner, during use of the robotic system 300, the motor(s) 312 may be used to advance the soft robotic tool 200 along a complex pathway, such as a complex anatomical pathway within the body of a patient, for carrying out a procedure, and to retract the tool 200 after completion of the procedure. In some embodiments, a plurality of motors 312 may be used, for example, with one motor 312 for advancing the soft robotic tool 200 and another motor 312 for retracting the tool 200. In other embodiments, a single motor 312 may be used for advancing and retracting the soft robotic tool 200.

The actuator(s) 314 may be configured to drive the tendons 230 of the soft robotic tool 200. In this manner, during use of the robotic system 300, the actuator(s) 314 may be used to pull one of the tendons 230 while allowing a remainder of the tendons 230 to assume the slack state, thereby causing the soft robotic tool 200 to articulate to assume various non-linear configurations as needed to navigate a complex pathway. In some embodiments, a plurality of actuators 314 may be used, for example, with the number of actuators 314 corresponding to the number of tendons 230. In this manner, each actuator 314 may be mechanically coupled to and configured to drive only one of the tendons 230. In other embodiments, a single actuator 314 may be used in conjunction with a mechanism for switching which tendon 230 is pulled at a particular time during a procedure.

The current generator(s) 316 may be configured to generate current for magnetizing the inductive coils of the magnetorheological fluid soft joints 220 of the soft robotic tool 200. In this manner, during use of the robotic system 300, the current generator(s) 316 may be used to selectively direct current to the inductive coils to switch the respective magnetorheological fluid soft joints 220 between the locked state and the unlocked state for allowing articulation of the soft robotic tool 200 about the unlocked magnetorheological fluid soft joint 220. In some embodiments, a plurality of current generators 316 may be used, for example, with the number of current generators 316 corresponding to the number of magnetorheological fluid soft joints 220. In this manner, each current generator 316 may be in electrical communication with and configured to magnetize the inductive coil of only one of the magnetorheological fluid soft joints 220. In other embodiments, a single current generator 316 may be used in conjunction with a mechanism for distributing current to the desired inductive coils of the magnetorheological fluid soft joints 220 at a particular time during a procedure.

The control unit(s) 320 may be configured to control operation of the motor(s) 312, the actuator(s) 314, and the current generator(s) 316 to facilitate desired movement and articulation of the soft robotic tool 200. In this manner, during use of the robotic system 300, the control unit(s) 320 may be used to selectively activate the motor(s) 312 for advancing and retracting the soft robotic tool 200, to selectively actuate the actuator(s) 314 for driving the tendons 230 of the tool 200, and to selectively cause the current generator(s) 316 to magnetize the inductive coils of the magnetorheological fluid soft joints 220 of the tool 200. In some embodiments, the control unit 320 may include a plurality of controllers for controlling operation of the motor(s) 312, the actuator(s) 314, and the current generator(s) 316. As shown, the control unit 320 may include one or more motor controller(s) 322, one or more actuator controller(s) 324, and one or more current controller(s) 326. The motor controller(s) 322 may be configured to control activation of the motor(s) 312 for advancing and retracting the soft robotic tool 200. The actuator controller(s) 324 may be configured to control actuation of the actuator(s) 314 for driving the tendons 230 of the soft robotic tool 200. In some embodiments, the actuator controller(s) 324 may be configured to cause only one of the tendons 230 to be pulled while a remainder of the tendons 230 are maintained in the slack state. The current controller(s) 326 may be configured to control the current generated by the current generator(s) 316 for magnetizing the inductive coils of the magnetorheological fluid soft joints 220 of the soft robotic tool 200. In this manner, the current controller(s) 326 may control a strength of a magnetic field generated by the respective inductive coils of the magnetorheological fluid soft joints 220. In some embodiments, the current controller(s) 326 may be configured to cause only one of the magnetorheological fluid soft joints 220 to assume the off state while a remainder of the joints 220 assume the on state. In some embodiments, the motor controller(s) 322, the actuator controller(s 324, and the current controller(s) 326 may be provided as separate, discrete controllers. In other embodiments, the motor controller(s) 322, the actuator controller(s) 324, and the current controller(s) 326 may be provided as portions or modules of a single controller. It will be appreciated that various configurations of the control unit 320 may be used to achieve the functions described above.

Although specific embodiments of the disclosure have been described, one of ordinary skill in the art will recognize that numerous other modifications and alternative embodiments are within the scope of the disclosure. For example, any of the functionality and/or processing capabilities described with respect to a particular device or component may be performed by any other device or component. Further, while various illustrative implementations and architectures have been described in accordance with embodiments of the disclosure, one of ordinary skill in the art will appreciate that numerous other modifications to the illustrative implementations and architectures described herein are also within the scope of this disclosure.

Although embodiments have been described in language specific to structural features and/or methodological acts, it is to be understood that the disclosure is not necessarily limited to the specific features or acts described. Rather, the specific features and acts are disclosed as illustrative forms of implementing the embodiments. Conditional language, such as, among others, “can,” “could,” “might,” or “may,” unless specifically stated otherwise, or otherwise understood within the context as used, is generally intended to convey that certain embodiments could include, while other embodiments do not include, certain features, elements, and/or steps. Thus, such conditional language is not generally intended to imply that features, elements, and/or steps are in any way required for one or more embodiments or that one or more embodiments necessarily include logic for deciding, with or without user input or prompting, whether these features, elements, and/or steps are included or are to be performed in any particular embodiment. The term “based at least in part on” and “based on” are synonymous terms which may be used interchangeably herein.

What is claimed is:

1. A soft robotic tool comprising:
a plurality of rigid links disposed in series;
a plurality of magnetorheological fluid soft joints, wherein each magnetorheological fluid soft joint is disposed between a pair of the rigid links, and wherein each magnetorheological fluid soft joint comprises:
a capsule containing a magnetorheological fluid; and
an inductive coil disposed around the capsule; and
a plurality of tendons extending along a length of the soft robotic tool, wherein each tendon is attached to each of the rigid links.

2. The soft robotic tool of claim 1, wherein each magnetorheological fluid soft joint is configured to assume an off state when no magnetic field is generated by the inductive coil and to assume an on state when a magnetic field is generated by the inductive coil, wherein each magnetorheological fluid soft joint is configured to allow articulation of the soft robotic tool about the magnetorheological fluid soft joint when the magnetorheological fluid soft joint is in the off state, and wherein each magnetorheological fluid soft joint is configured to inhibit articulation of the soft robotic tool about the magnetorheological fluid soft joint when the magnetorheological fluid soft joint is in the on state.

3. The soft robotic tool of claim 1, wherein the rigid links are formed of a polymeric material, a metallic material, or a ceramic material.

4. The soft robotic tool of claim 1, wherein the capsule is formed of a polymeric material, and wherein the magnetorheological fluid comprises a dispersion of magnetic particles in a non-conductive, non-magnetic carrier fluid.

5. The soft robotic tool of claim 1, wherein the plurality of tendons comprises a first tendon and a second tendon, wherein the rigid links define a first tendon routing pathway and a second tendon routing pathway, wherein the first tendon extends along the first tendon routing pathway, and wherein the second tendon extends along the second tendon routing pathway.

6. The soft robotic tool of claim 5, wherein the plurality of rigid links comprises a first rigid link and a second rigid link, wherein the first rigid link defines a first portion of the first tendon routing pathway and a first portion of the second tendon routing pathway, and wherein the second rigid link defines a second portion of the first tendon routing pathway and a second portion of the second tendon routing pathway.

7. The soft robotic tool of claim 5, wherein the plurality of magnetorheological fluid soft joints comprises a first magnetorheological fluid soft joint and a second magnetorheological fluid soft joint, wherein the first tendon and the second tendon are configured to bend the first magnetorheological fluid soft joint in a first bending plane, and wherein the first tendon and the second tendon are configured to bend the second magnetorheological fluid soft joint in a second bending plane transverse to the first bending plane.

8. The soft robotic tool of claim 5, wherein the plurality of magnetorheological fluid soft joints comprises a first magnetorheological fluid soft joint and a second magnetorheological fluid soft joint, wherein the first tendon and the second tendon are configured to bend the first magnetorheological fluid soft joint in a bending plane, and wherein the first tendon and the second tendon are configured to bend the second magnetorheological fluid soft joint in the bending plane.

9. The soft robotic tool of claim 1, wherein the plurality of rigid links comprises a first rigid link, a second rigid link, and a third rigid link, wherein each tendon is movably attached to each of the first rigid link and the second rigid link, and wherein each tendon is fixedly attached to the third rigid link.

10. A robotic system comprising:
a soft robotic tool comprising:
a plurality of rigid links disposed in series;
a plurality of magnetorheological fluid soft joints, wherein each magnetorheological fluid soft joint is disposed between a pair of the rigid links, and wherein each magnetorheological fluid soft joint comprises:
a capsule containing a magnetorheological fluid; and
an inductive coil disposed around the capsule; and
a plurality of tendons extending along a length of the soft robotic tool, wherein each tendon is attached to each of the rigid links;
an actuation module comprising:
a motor configured to advance and retract the soft robotic tool relative to the actuation module; and
a plurality of actuators configured to drive the tendons, wherein each actuator is coupled to at least one of the tendons.

11. The robotic system of claim 10, wherein each magnetorheological fluid soft joint is configured to assume an off state when no magnetic field is generated by the inductive coil and to assume an on state when a magnetic field is generated by the inductive coil, wherein each magnetorheological fluid soft joint is configured to allow articulation of the soft robotic tool about the magnetorheological fluid soft joint when the magnetorheological fluid soft joint is in the off state, and wherein each magnetorheological fluid soft joint is configured to inhibit articulation of the soft robotic tool about the magnetorheological fluid soft joint when the magnetorheological fluid soft joint is in the on state.

12. The robotic system of claim 11, wherein the actuation module further comprises a motor controller configured to control activation of the motor for advancing and retracting the soft robotic tool.

13. The robotic system of claim 11, wherein the actuation module further comprises an actuator controller configured to control activation of the actuators for driving the tendons to articulate the soft robotic tool about the magnetorheological fluid soft joints, and wherein the actuator controller is configured to cause only one of the tendons to be pulled while a remainder of the tendons are maintained in a slack state.

14. The robotic system of claim 11, wherein the actuation module further comprises a plurality of current controllers in communication with the inductive coils of the magnetorheological fluid soft joints, wherein each current controller is configured to control a strength of a magnetic field generated by one of the inductive coils, and wherein the current controllers are configured to cause only one of the magnetorheological fluid soft joints to assume the off state while a remainder of the magnetorheological fluid soft joints assume the on state.

15. The robotic system of claim 10, further comprising one or more surgical tools mounted to the soft robotic tool.

16. The robotic system of claim 10, wherein the plurality of rigid links comprises a first rigid link, a second rigid link, and a third rigid link, wherein the plurality of tendons comprises a first tendon and a second tendon, wherein the first tendon is movably attached to each of the first rigid link and the second rigid link by passing through respective apertures defined by the first rigid link and the second rigid link, wherein the first tendon is fixedly attached to the third rigid link, wherein the second tendon is movably attached to each of the first rigid link and the second rigid link by passing through respective apertures defined by the first rigid link and the second rigid link, and wherein the second tendon is fixedly attached to the third rigid link.

17. The robotic system of claim 16, wherein the first rigid link is disposed at a proximal end of the soft robotic tool, wherein the third rigid link is disposed at a distal end of the soft robotic tool, and wherein the second rigid link is disposed between the first rigid link and the third rigid link.

18. A soft robotic tool comprising:
a plurality of rigid links disposed in series; and
a plurality of magnetorheological fluid soft joints, wherein each magnetorheological fluid soft joint is disposed between a pair of the rigid links, and wherein each magnetorheological fluid soft joint comprises:
a capsule containing a magnetorheological fluid; and
an inductive coil disposed around the capsule.

19. The soft robotic tool of claim 18, wherein each magnetorheological fluid soft joint is configured to assume an off state when no magnetic field is generated by the inductive coil and to assume an on state when a magnetic field is generated by the inductive coil, wherein each magnetorheological fluid soft joint is configured to allow articulation of the soft robotic tool about the magnetorheological fluid soft joint when the magnetorheological fluid soft joint is in the off state, and wherein each magnetorheological fluid soft joint is configured to inhibit articulation of the soft robotic tool about the magnetorheological fluid soft joint when the magnetorheological fluid soft joint is in the on state.

20. The soft robotic tool of claim 18, wherein the capsule is formed of a polymeric material, and wherein the magnetorheological fluid comprises a dispersion of magnetic particles in a non-conductive, non-magnetic carrier fluid.
